SEQUENCE LISTING (8 sequences)

SEQ ID NO: 1 - DNA, 48 bp, Artificial
  Synthetic
aatccgtcgagcagagttagttagggttagggttagggttagggttag

SEQ ID NO: 2 - DNA, 18 bp, Artificial
  Synthetic
aatccgtcgagcagagtt

SEQ ID NO: 3 - DNA, 30 bp, Artificial
  Synthetic
gcgcggcttacccttacccttaccctaacc

SEQ ID NO: 4 - DNA, 44 bp, Artificial
  Synthetic
aatccgtcgagcagagttagggttagggttagggttagggttag

SEQ ID NO: 5 - RNA, 11 nt, Homo sapiens
cuaacccuaac

SEQ ID NO: 6 - DNA, 26 bp, Artificial
  Synthetic
aatccgtcgagcagagttagggttag

SEQ ID NO: 7 - DNA, 49 bp, Artificial
  Synthetic
aatccgtcgagcagagttagggttagggttagggttagggttagggtta

SEQ ID NO: 8 - DNA, 50 bp, Artificial
  Synthetic
gcgcggcttacccttacccttaccctaaccctaactctgctcgacggatt

(19) United States
(12) Patent Application Publication
(10) Pub. No.: US 2014/0106359 A1
(43) Pub. Date: Apr. 17, 2014

(54) DIGITAL TELOMERASE ASSAY

(71) Applicant: BIO-RAD LABORATORIES, INC., HERCULES, CA (US)

(72) Inventors: Claudia Litterst, Walnut Creek, CA (US); Ryan Thane Koehler, West Linn, OR (US)

(73) Assignee: BIO-RAD LABORATORIES, INC., HERCULES, CA (US)

(21) Appl. No.: 14/054,698

(22) Filed: Oct. 15, 2013

Related U.S. Application Data
(60) Provisional application No. 61/714,033, filed on Oct. 15, 2012.

(51) Int. Cl. C12Q 1/48

(52) U.S. Cl. CPC C12Q 1/48
USPC 435/6.12

(57) ABSTRACT

Digital assay system, including methods and apparatus, for determining a presence or activity of telomerase in a sample.

### CROSS-REFERENCE TO PRIORITY APPLICATION

This application is based upon and claims the benefit under 35 U.S.C. §119(e) of U.S. Provisional Patent Application Ser. No. 61/714,033, filed Oct. 15, 2012, which is incorporated herein by reference in its entirety for all purposes.

### CROSS-REFERENCES TO OTHER MATERIALS

This application incorporates by reference in their entireties for all purposes the following materials: U.S. Pat. No. 7,041,481, issued May 9, 2006; U.S. Patent Application Publication No. 2010/0173394 A1, published Jul. 8, 2010; U.S. Patent Application Publication No. 2011/0217712 A1, published Sep. 8, 2011; U.S. Patent Application Publication No. 2012/0152369 A1, published Jun. 21, 2012; and Joseph R. Lakowicz, PRINCIPLES OF FLUORESCENCE SPECTROSCOPY (2^{nd} Ed. 1999).

### INTRODUCTION

Telomeres are the natural ends of a linear chromosome that serve to stabilize the chromosome. Each telomere is generally composed of a highly repetitive DNA sequence. For example, in vertebrates, such as humans, a hexamer repeat (5′-TTAGGG-3′) is present hundreds or thousands of times in tandem at each chromosome end to generate telomeres that are kilobases in length.

Telomeres also serve as binding sites for proteins that help maintain chromosomal integrity. The proteins prevent end-to-end joining, degradation, and recombination, among others, at the ends of chromosomes.

DNA polymerase cannot replicate the very end of a linear chromosome (e.g., the last 100 to 200 nucleotides or so) due to the lagging-strand problem. As a result, in most cell types, each telomere decreases in length every replication cycle, which is linked to the mitotic clock that limits the number of cell divisions permitted before a mortal cell undergoes senescence. Telomere length can serve as an indicator of cell age.

Immortal cells, such as tumor cells and stem cells, can avoid this progressive telomere shortening through the action of an enzyme, telomerase, which adds copies of the basic telomere repeat to maintain or even increase telomere length as the immortal cells proliferate. Telomerase is a ribonucleoprotein that utilizes an integral reverse transcriptase activity, and an RNA template for the basic repeat, to extend the leading strand at the end of a telomere. The extended portion of the leading strand then functions as a template for lagging strand synthesis to produce a double-stranded extension of the telomere.

Telomerase activity in a cell may not be well correlated with the level of telomerase expression. For example, cell lysates that exhibit little or no detectable telomerase activity may contain substantial amounts of telomerase messenger RNA. Therefore, telomerase measurements that are most meaningful to the researcher and/or clinician reflect the ability to counter the mitotic clock.

Measurement of activity levels of telomerase obtained from normal and diseased cells can provide insight into stem cell behavior, cell immortalization, tumor vulnerability, and the like. Accordingly, researchers and clinicians would welcome more options for monitoring telomerase; a new telomerase assay is needed.

### SUMMARY

The present disclosure provides a digital assay system, including methods and apparatus, for determining a presence or activity of telomerase in a sample.

### BRIEF DESCRIPTION OF THE DRAWINGS

FIG. 1 is a flowchart of steps that may be performed in an exemplary method of measuring a presence or activity of telomerase in a sample with a digital assay system, in accordance with aspects of the present disclosure.

FIG. 2 is a schematic illustration of exemplary configurations that may be produced during performance of the method of FIG. 1 to measure an activity of telomerase in a sample, in accordance with aspects of the present disclosure.

FIG. 3 is a schematic illustration of exemplary configurations that may be produced during performance of the method of FIG. 1 to measure a concentration of active telomerase, in accordance with aspects of the present disclosure.

FIG. 4 is a schematic illustration of an exemplary digital assay system for measuring a presence or activity of telomerase, in accordance with aspects of the present disclosure.

FIG. 5 is a schematic illustration of exemplary configurations that may be produced during product generation in the method of FIG. 1, in accordance with aspects of the present disclosure.

FIG. 6 is an amplification scheme for amplifying at least a region of the product of FIG. 5 with a pair of primers, in accordance with aspects of the present disclosure.

FIG. 7 is a plot of exemplary photoluminescence data collected from droplets after performing amplification of a region of a template (or a no-template control (NTC)) with the primers of FIG. 6 at various annealing temperatures, with the template being present at limiting dilution in the droplets, synthesized chemically, and corresponding to a truncated version of the product of FIGS. 5 and 6 containing only about four hexamer repeats.

FIG. 8 is a plot of template concentrations calculated from the data of FIG. 7.

### DETAILED DESCRIPTION

The present disclosure provides a digital assay system, including methods and apparatus, for determining a presence or activity of telomerase in a sample.

An exemplary method of assaying telomerase is provided. In the method, a product may be generated by extending a substrate with telomerase. Partitions containing at least a portion of the telomerase may be formed. An amplification reaction may be performed in partitions with the product as a template. The product may be present in partitions at limiting dilution before the application reaction is initiated. Data may be collected for the amplification reaction from a plurality of the partitions. A level of the product and/or a presence or activity of telomerase may be determined based on the data.

The digital assays of the present disclosure for assaying telomerase may have numerous advantages over other approaches, including any combination of higher throughput, better accuracy and/or sensitivity, reduced use of consumables, and more or different information about active telomerase.

Further aspects of the present disclosure are presented in the following sections: (I) overview of digital telomerase assays, and (II) examples.

### I. OVERVIEW OF DIGITAL TELOMERASE ASSAYS

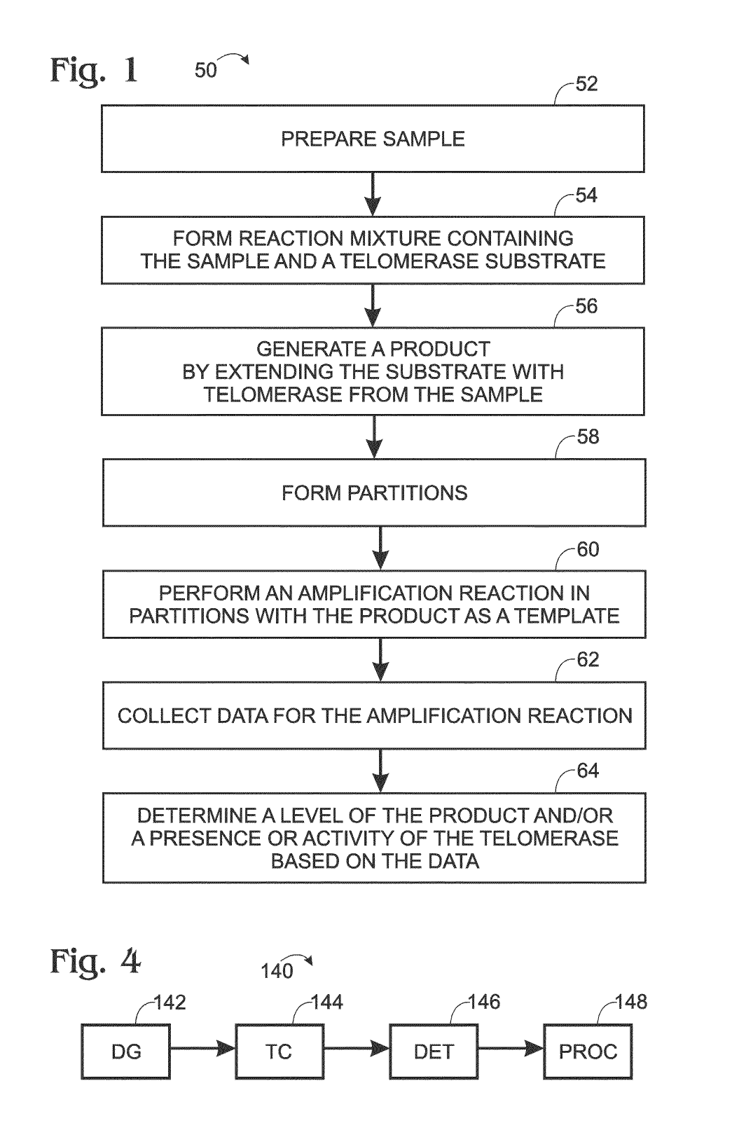

This section provides an overview of a digital assay system for measuring a presence or activity of telomerase provided by a sample; see FIGS. 1-4.

Digital assays generally rely on the ability to detect the presence or activity of individual copies of an analyte, such as a nucleic acid template (also termed a target), in a sample. In an exemplary digital assay, portions of a sample are separated into a set of partitions, generally of equal volume, with each containing, on average, about one copy of the analyte. If the copies of the analyte are distributed randomly among the partitions, some partitions should contain no copies, others only one copy, and, if the number of partitions is large enough, still others should contain two copies, three copies, and even higher numbers of copies. The probability of finding exactly 0, 1, 2, 3, or more copies in a partition, based on a given average concentration of analyte in the partitions, may be described by a Poisson distribution. Conversely, the concentration of analyte in the partitions (and thus in the sample) may be estimated from the probability of finding a given number of copies in a partition.

Estimates of the probability of finding no copies and of finding one or more copies may be measured in the digital assay. Each partition can be tested to determine whether the partition is a positive partition that contains at least one copy of the analyte, or is a negative partition that contains no copies of the analyte. The probability of finding no copies in a partition can be approximated by the fraction of partitions tested that are negative (the “negative fraction”), and the probability of finding at least one copy by the fraction of partitions tested that are positive (the “positive fraction”). The negative fraction (or, equivalently, the positive fraction) then may be utilized to determine the concentration of the analyte in the partitions by Poisson statistics.

Digital assays frequently rely on amplification of a nucleic acid template in partitions, such as droplets, to enable detection of a single copy of the template. In some cases, amplification may, for example, be conducted via the polymerase chain reaction (PCR), to achieve a digital PCR assay. In any event, amplification of the template to generate a corresponding amplicon in individual partitions can be detected optically in the presence of a reporter, such as a sequence-specific probe or a more generic DNA-binding dye (e.g., an intercalating dye), included in the reaction. The sequence-specific probe may include an optical label that provides a photoluminescence signal (e.g., fluorescence or phosphorescence) indicating whether or not the template has been amplified in each partition.

FIG. 1 shows a flowchart of steps that may be performed in an exemplary method 50 of assaying telomerase. The steps listed may be performed in any suitable order, in any suitable combination, and may be combined with or modified by any other suitable aspects of the present disclosure.

Sample Preparation.

A sample may be prepared for analysis in the assay, indicated at 52. Preparation of the sample may include any suitable manipulation of the sample that preserves telomerase activity, such as collection, dilution, concentration, purification, combination with one or more assay reagents, or any combination thereof, among others.

In some cases, preparation of the sample may include lysing cells. Exemplary lysis procedures include breaking cells with a hypotonic lysis solution and/or a lysis solution containing a non-ionic detergent. Lysis also or alternatively may be encouraged mechanically, such as with a Dounce homogenizer, sonication, vortexing, or the like.

The sample may be obtained from any suitable source of telomerase. Exemplary sources include one or more cells (e.g., eukaryotic cells), such as a single cell, a group of cells (e.g., cultured cells, cells isolated from biological fluid, or the like), a tissue sample, a biological fluid (e.g., blood, lymph, urine, semen, tears, mucus, saliva, etc.), a whole organism, or the like. Exemplary organisms that may be suitable include a fungus (e.g., Aspergillus or Saccharomyces cerevisiae), a nematode (e.g., C. elegans), an insect (e.g., Drosophila melanogaster), a fish (e.g., zebrafish), a frog (e.g., Xenopus laevis), a mammal (e.g., humans), or the like. The sample may be a research sample, an environmental sample, a clinical sample, or a forensic sample, among others.

Reaction Mixture Formation.

A reaction mixture containing the sample and a telomerase substrate may be formed, indicated at 54. The reaction mixture may be configured to permit active telomerase, if any, in the sample to extend the substrate. Accordingly, the reaction mixture may include dNTPs (+/−dCTP), buffer, salt, a surfactant, and the like. The reaction mixture may include any suitable portion or all of the sample.

In some embodiments, formation of the reaction mixture may include combining the sample with reagents for amplification and for reporting whether or not amplification occurred. Reagents for amplification may include any combination of primers for one or more templates (e.g., one or more primers to amplify at least a region of the product), dNTPs and/or NTPs, at least one enzyme (e.g., a polymerase, a ligase, or a combination thereof, each of which may or may not be heat-stable), and/or the like. Accordingly, the reaction mixture may be capable of amplification of each of one or more types of template, if present or produced, in the reaction mixture (or a partition thereof). Formation of the reaction mixture may render the reaction mixture capable of reporting, or being analyzed for, whether or not amplification has occurred, for each template, and optionally the extent of any such amplification.

The reaction mixture may include a reporter. The reporter may interact at least generally nonspecifically or specifically with an amplicon generated by an amplification reaction. In some cases, the reporter may be a generic reporter having a general affinity for nucleic acid (single and/or double-stranded) without substantial sequence-specific binding. In some cases, the reporter may be a labeled probe that includes a nucleic acid (e.g., an oligonucleotide) labeled with a luminophore, such as a fluorophore or phosphor, among others.

The reaction mixture may be formed under conditions that discourage formation of product from the substrate. For example, the reaction mixture and/or components thereof may be kept on ice, and the reaction mixture may not be completed until the user is ready to initiate (and time) product generation. In this way, product generation can be performed under standardized conditions, which allows the amount of generated product to be compared among samples.

The telomerase substrate may have any suitable characteristics. The substrate may be an oligonucleotide that can be extended by active telomerase. The oligonucleotide may be DNA or RNA, may be synthetic, and/or may have any suitable length, such as about 10 to 200 nucleotides or about 15 to 100 nucleotides, among others. Also, the substrate may have a 3′-end sequence that is complementary to an RNA template of the telomerase to be assayed. Exemplary 3′-end sequences that may be suitable include 5′-GT-3′, 5′-GTT-3′, 5′-GTTA-3′, 5′-GTTAG-3′, 5′-GTTAGG-3′, 5′-TTA-3′, 5′-TTAG-3′, 5′-TTAGG-3′, or the like. The substrate may include a primer sequence (for later amplification with a corresponding primer and with the product as a template), a probe sequence (to enable binding of a probe to at least a region of an amplicon during and/or after amplification), or a combination thereof. If the substrate provides both a primer sequence and a probe sequence, the probe sequence may or may not overlap the primer sequence. In some cases, the probe sequence may be located 3′ of the primer sequence.

Product Generation.

A product may be generated from the substrate with telomerase provided by the sample, indicated at 56. The telomerase may extend the substrate by any suitable length, to form a product of any suitable length (generally, a variable length). The telomerase may add a plurality of repeats, such as hexamer repeats for a vertebrate telomerase. The number of repeat copies added may be determined by the activity state of the telomerase, the availability of dNTPs, the amount of substrate present, the temperature at which product is generated, the length of time during which product is generated, or a combination thereof, among others. In exemplary embodiments, the product is generated by incubating the reaction mixture at a defined temperature for a defined length of time. Product generation can be terminated by inactivating telomerase, such as by heat-killing the telomerase or addition of a telomerase inhibitor. The product may be generated, at least in part or at least predominantly, before or after partitions are formed.

Each copy of active telomerase may form any suitable number of product molecules. The ratio of active telomerase copies to product molecules formed may be about, less than about, or greater than about 1:1, 1:2, 1:5, 1:10, 1:100, or 1:1000, among others. In some cases, product may be generated under conditions that discourage an active telomerase copy from forming more than one molecule of product. Such conditions may include a low reaction temperature (e.g., 4° C., 10° C., 15° C., or 20° C., among others), a short reaction time (e.g., less than about 10 seconds, 30 seconds, or 1 minute, among others), or the like.

Partition Formation.

The sample, reaction mixture, and/or product may be divided or separated into partitions, indicated at 58. Formation of partitions may involve distributing any suitable portion or all of the sample, reaction mixture, and/or product to the partitions. Each partition may be and/or include a fluid volume that is isolated from the fluid volumes of other partitions. The partitions may be isolated from one another by an immiscible fluid phase, such as a carrier phase (interchangeably termed a continuous phase) of an emulsion, by a solid phase, such as at least one wall of a container, or a combination thereof, among others. In some embodiments, the partitions may be droplets disposed in a continuous phase, such that the droplets and the continuous phase collectively form an emulsion.

The partitions may be formed by any suitable procedure, in any suitable manner, and with any suitable properties. For example, the partitions may be formed with a fluid dispenser, such as a pipette, with a droplet generator, by agitation (e.g., shaking, stirring, sonication, etc.) of the sample/reaction mixture/product, and/or the like. Accordingly, the partitions may be formed serially, in parallel, or in batch. The partitions may have any suitable volume or volumes. The partitions may be have a substantially uniform volume or may have different volumes. Exemplary partitions having substantially the same volume are monodisperse droplets. Exemplary volumes for the partitions include an average volume of less than about 100, 10 or 1 μL, less than about 100, 10, or 1 nL, or less than about 100, 10, or 1 μL, among others.

The partitions, when created, may be competent for performance of one or more reactions in the partitions. Alternatively, partition formation may include modifying partitions to add one or more reagents after the partitions are created to render the partitions competent for reaction. The reagents may be added by any suitable mechanism, such as a fluid dispenser, fusion of partitions (e.g., merging droplets), or the like.

The partitions may be formed with any suitable concentration (i.e., copies/molecules per partition) of product and/or active telomerase. The partitions when formed and/or before initiation of an amplification reaction may contain the product (and/or the telomerase) at “limiting dilution,” which means that one or more of the partitions contain no molecules/copies of the product (and/or telomerase), one or more of the partitions contain a single molecule/copy (only one molecule/copy) of the product (and/or telomerase), and, optionally, one or more of the partitions (e.g., the rest of the partitions) may contain two or more molecules/copies of the product (and/or telomerase). The term “limiting dilution” permits but does not require a literal dilution of fluid containing the product (and/or telomerase), and is not restricted to the case where there is no more than one molecule/copy of the product (and/or telomerase) in any partition. Accordingly, partitions containing the product (and/or telomerase) at limiting dilution may, for example, contain an average of more than, or less than, about one molecule/copy, two molecules/copies, or three molecules/copies, among others, of the product (and/or telomerase) per partition when the partitions are formed and/or before an amplification reaction is initiated. Molecules/copies of the product (and/or telomerase) may have a random distribution among the partitions, which may be described as a Poisson distribution.

Product Amplification.

An amplification reaction may be performed in the partitions with the product as a template, indicated at 60. The amplification reaction may amplify one or more regions of the product and may generate/amplify an amplicon. The amplicon may correspond to a region of the product (e.g., a product region of fixed or variable length). Amplification of region of the product interchangeably may be described as amplification of an amplicon representing or corresponding to the product. The amplicon may have a fixed or variable length among and/or within partitions.

Amplification may occur substantially in only a subset of the partitions, such as less than about nine-tenths, three-fourths, one-half, one-fourth, or one-tenth of the partitions, among others. In some examples, the amplification reaction may be a polymerase chain reaction and/or ligase chain reaction. Accordingly, a plurality of amplification reactions for a plurality of distinct types of products and/or targets may be performed simultaneously in the partitions.

Amplification may or may not be performed isothermally. In some cases, amplification in the partitions may be encouraged by heating the partitions and/or incubating the partitions at a temperature above room temperature, such as at a denaturation temperature, an annealing temperature, and/or an extension temperature. In some examples, the conditions may include thermally cycling the partitions to promote a polymerase chain reaction and/or ligase chain reaction. Exemplary isothermal amplification approaches that may be suitable include nucleic acid sequence-based amplification, transcription-mediated amplification, multiple-displacement amplification, strand-displacement amplification, rolling-circle amplification, loop-mediated amplification of DNA, helicase-dependent amplification, and single-primer amplification, among others.

Data Collection.

Data, such as luminescence data, may be collected from partitions, indicated at 62. Data collection may include creating one or more signals representative of light detected from the partitions. The signals may represent a property of light, such as the intensity, polarization, and/or lifetime of light, emitted from the partitions in response to illumination with excitation light. The signals may be created based on detected light emitted from a reporter in the partitions. Exemplary reporters include a generally nonspecific (generic) DNA-binding dye (e.g., an intercalating dye), a dye-labeled oligonucleotide probe, or the like.

Partitions may be analyzed and signals created at any suitable time(s). Exemplary times include at the end of an assay (endpoint assay), when reactions have run to completion and the data no longer are changing, or at some earlier time, as long as the data are sufficiently and reliably separated.

Determination of Presence, Level, and/or Activity.

A level of the product and/or a presence or activity of telomerase may be determined, indicated at 64. The level of product may be relative or absolute, and may be a concentration. In exemplary embodiments, a level of the product in the partitions is determined from the collected data, and then the presence of activity of telomerase is determined based on the level of the product.

Amplification of product in individual partitions may be ascertained based on the collected data. A signal detected from the reporter in the partitions may be analyzed to determine whether or not at least one molecule of the product is present in each given partition. A number of partitions that are positive for the product and/or a number of partitions that are negative for the product may be determined based on the data, such as by counting partitions deemed to be amplification positive or counting partitions deemed to be amplification negative. The signal detected from each partition, and the partition itself, may be classified as being positive or negative for the product. Classification may be based on the amplitude of light detected from each partition. If the signal/partition is classified as positive (+) for the product, amplification of the product is deemed to have occurred and at least one molecule of the product is deemed to have been present in the partition before amplification. In contrast, if the signal/partition is classified as negative (−), amplification of the product is deemed not to have occurred and the product is deemed to be absent from the partition.

A measure representative of a level of the product may be determined. The level may be determined based on the number of partitions that are amplification-positive for the product. The calculation may be based on molecules of the product having a Poisson distribution among the partitions. The measure may be a relative level of the product, such as a ratio of the level of the product to another target. The total number of partitions (from which data was collected) may be counted or, in some cases, estimated.

An absolute level (e.g., a concentration) of the product may be determined. A fraction of the total number of partitions that are negative (or, equivalently, positive) for the product may be calculated. The fraction may be calculated as the number of counted negative (or, equivalently, positive) partitions for the product divided by the total number of partitions analyzed.

The concentration of the product may be obtained. The concentration may be expressed with respect to the partitions, and/or with respect to a sample disposed in the partitions and serving as the source of telomerase. The concentration of the product in the partitions may be calculated from the fraction of positive partitions by assuming that product molecules have a Poisson distribution among the partitions. With this assumption, the fraction f(k) of partitions having k molecules of the product is given by the following equation:

$$f ( k ) = \frac{{c}^{k}}{k !} {e}^{- C} \qquad ( 1 )$$

Here, C is the concentration of the product in the partitions, expressed as the average number of product molecules per partition. Simplified Poisson equations may be derived from the more general equation above and may be used to determine product concentration from the fraction of positive partitions. An exemplary Poisson equation that may be used is as follows:

$$C = - ln ( 1 - \frac{{N}_{+}}{{N}_{tot}} ) \qquad ( 2 )$$

where N₊ is the number of positive partitions and Nₜₒₜ is the total number of partitions, such that N₊/Nₜₒₜ is equal to fₚ, which is the fraction of partitions positive for the product (i.e., fₚ=f(1)+f(2)+f(3)+ . . . ), and which is a measured estimate of the probability of a partition having at least one molecule of the product. Another exemplary Poisson equation that may be used is as follows:

$$C = - ln ( \frac{{N}_{0}}{{N}_{tot}} ) \qquad ( 3 )$$

where N₀ is the number of negative partitions and Nₜₒₜ is the total number of partitions, such that N₀/Nₜₒₜ is equal to fₙ, which is the fraction of negative partitions (or 1−fₚ), which is a measured estimate of the probability of a partition having no molecules of the product, and C is the concentration as described above.

In some embodiments, an estimate of the concentration of the product may be obtained directly from the positive fraction, without use of Poisson statistics. In particular, the positive fraction and the concentration converge as the concentration decreases. For example, with a positive fraction of 0.1, the concentration is determined with the above equation to be about 0.105, a difference of only 5%; with a positive fraction of 0.01, the concentration is determined to be about 0.01005, a ten-fold smaller difference of only 0.5%. However, use of Poisson statistics can provide a more accurate estimate of concentration, particularly with a relatively higher positive fraction, because Poisson statistics accounts for the occurrence of multiple product molecules per partition.

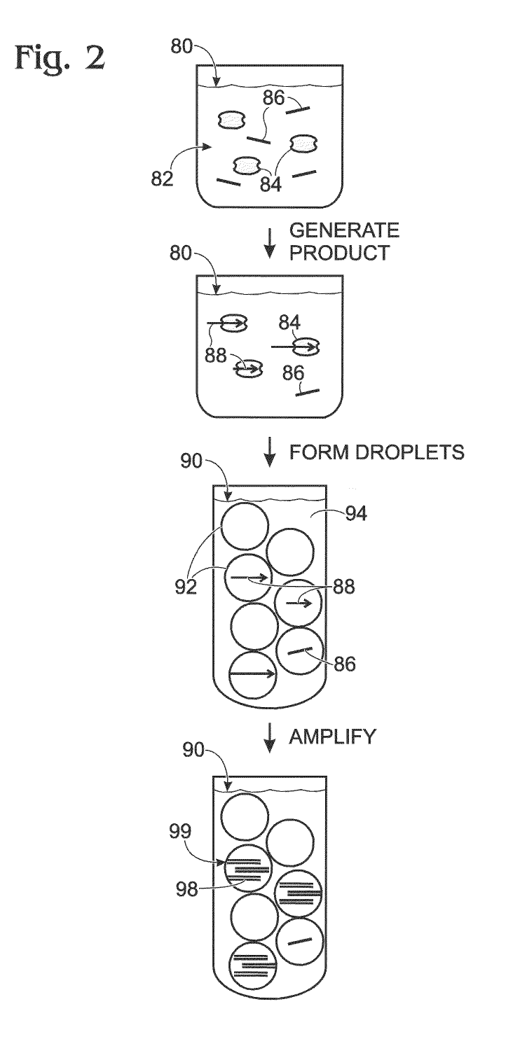

FIG. 2 shows exemplary configurations that may be produced during performance of method 50 of FIG. 1 to measure telomerase activity. In the depicted embodiment, the partitions are droplets.

A reaction mixture 80 may be formed. The reaction mixture may contain a sample 82, telomerase 84 provided by the sample, and a substrate 86 for the telomerase. The reaction mixture may be considered a bulk phase. The reaction mixture also may contain any other suitable reagents, such as salt, buffer, detergent, dNTPs (or dATP, dGTP, and dTTP, but not dCTP), and the like. The reaction mixture may or may not contain reagents sufficient for subsequent amplification of the extended substrate.

A product 88 may be generated from substrate 86 by the action of telomerase 84. The product may include a plurality of tandem repeats added by telomerase 84 (and indicated by an arrowhead). A plurality of substrate copies in the reaction mixture may fail to be extended by telomerase. Product 88 may be generated by maintaining the reaction mixture at an appropriate temperature for an appropriate length of time. For example, the reaction mixture may be placed at 20° C., 25° C., 30° C., or 37° C., among others, for about 1, 2, 5, 10, 20, or 60 minutes, among others. Product generation may be terminated by heat-killing the telomerase, such as by incubating the reaction mixture at about 90° C. for one minute.

An emulsion 90 may be formed containing molecules of product 88. The emulsion has a plurality of droplets 92 disposed in a carrier phase 94 (interchangeably termed a continuous phase). Product 88 may be present in the droplets at limiting dilution. For example, in the present illustration, only about one-half of the droplets contain at least one molecule of the product. In other embodiments, about 10% to 90% of the droplets may contain at least one molecule of the product. Each droplet also may include reagents for product amplification, such as one or more amplification primers, dNTPs (or NTPs), a polymerase and/or ligase, a reporter for amplification, and the like.

Amplification may be performed in droplets 92 to produce an amplicon 98 in each amplification-positive droplet 99. The presence of amplicon 98 in a droplet indicates the presence of at least one molecule of product 88 in the droplet before amplification. Amplification-positive and amplification-negative droplets then may be distinguished with amplification data collected from the droplets.

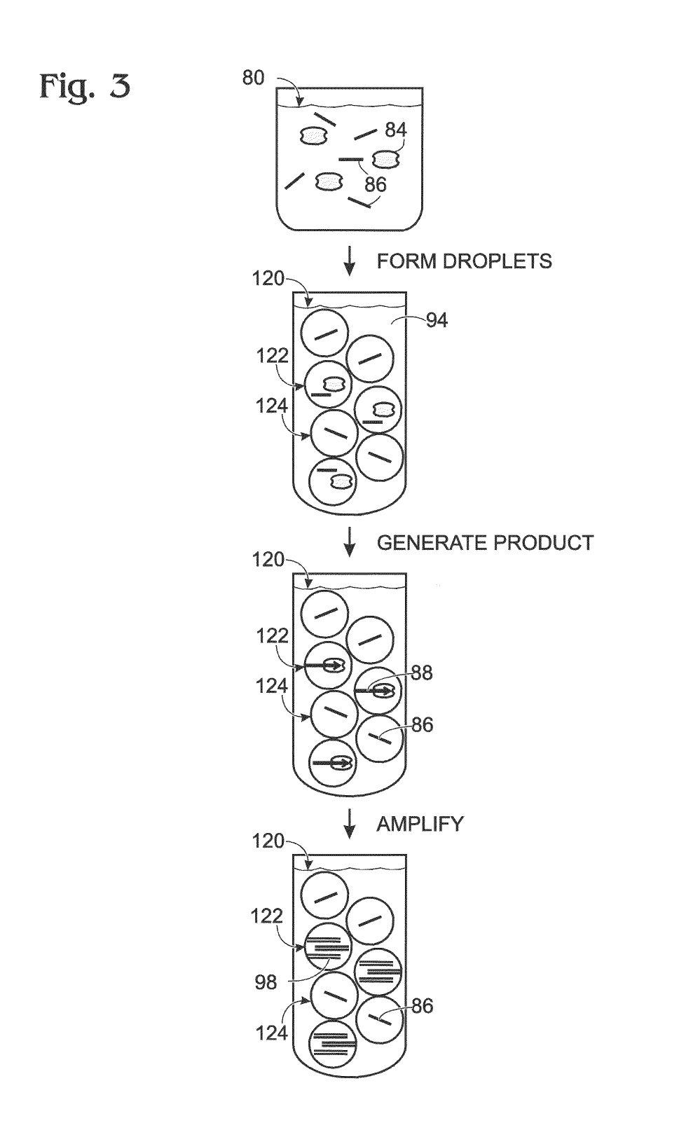

FIG. 3 shows exemplary configurations that may be produced during performance of method 50 of FIG. 1 to measure a level (e.g., a concentration) of active telomerase. In the depicted embodiment, the partitions are droplets. Reaction mixture 80 may be formed with all of the reagents needed for product generation and subsequent amplification of the product. One or more of the amplification reagents, such as a polymerase, may be inactive or unavailable for reaction until activated or released, to reduce the incidence of undesired side reactions before amplification. Also, the reaction mixture may be manipulated under conditions that minimize telomerase activity, such as being maintained on ice before droplet formation.

The reaction mixture may be partitioned to form an emulsion of droplets in a carrier phase 94. The droplets may be formed at a limiting dilution of active telomerase. In particular, only a subset of the droplets, such as droplet 122, contain a copy of active telomerase, while another subset of the droplets, such as droplet 124, do not contain active telomerase.

Product 88 is then generated in the droplets by extension of substrate 86 with telomerase 84. Only droplets containing an active copy of telomerase, such as droplet 122, are able to form the product.

At least a region of the product is then amplified in the droplets to produce amplicon 98. Amplification-positive droplets (e.g., droplet 122) contain amplicon 98 and amplification-negative droplets (e.g., droplet 124) do not. Each amplification-positive droplet contains at least one copy of active telomerase. Accordingly, determining the fraction of droplets that are negative (or positive) for the product allows the concentration of product (e.g., as molecules per droplet) to be determined, which is substantially equal to the concentration of active telomerase.

FIG. 4 shows an exemplary system 140 for performing any suitable combination of steps of the digital assay of FIG. 1. System 140 may include a partitioning assembly, such as a droplet generator 142 (“DG”), a thermal incubation assembly, such as a thermocycler 144 (“TC”), a detection assembly (a detector) 146 (“DET”), and a data processing assembly (a processor) 148 (“PROC”), or any combination thereof, among others. The data processing assembly may be, or may be included in, a controller that communicates with and controls operation of any suitable combination of the assemblies. The arrows between the assemblies indicate movement or transfer of material, such as fluid (e.g., a continuous phase of an emulsion) and/or partitions (e.g., droplets) or signals/data, between the assemblies. Any suitable combination of the assemblies may be operatively connected to one another, and/or one or more of the assemblies may be unconnected to the other assemblies, such that, for example, material/data is transferred manually.

System 140 may operate as follows. Droplet generator 142 may form droplets disposed in a continuous phase. The droplets may be cycled thermally with thermocycler 144 to promote amplification of product in the droplets. Data may be collected from the droplets with detector 146. The data may be processed by processor 148 to determine numbers of droplets (e.g., a total number and a number amplification-positive and/or amplification-negative for the product), a product level, a telomerase activity, and/or a telomerase concentration, among others.

Further aspects of sample preparation, droplet generation, data collection, and target (e.g., product) level determination, among others, that may be suitable for the system of the present disclosure are described in the references listed above under Cross-References, which are incorporated herein by reference.

### II. EXAMPLES

The following examples describe selected aspects and embodiments of digital telomerase assays. These examples are intended for illustration only and should not limit the entire scope of the present disclosure.

### Example 1

### Exemplary Reagents and Data for Product Amplification

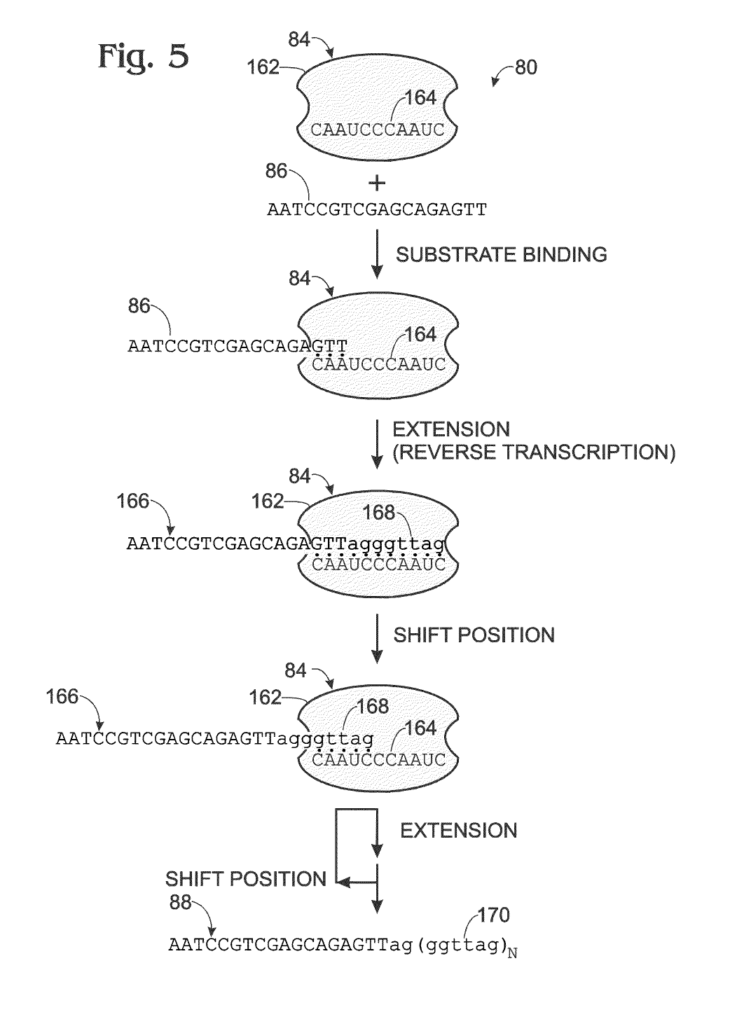

This example describes further aspects of the telomerase assay of FIG. 1, including exemplary amplification results obtained with a chemically-synthesized template corresponding to a product generated by telomerase extension of a substrate; see FIGS. 5-8.

FIG. 5 shows exemplary configurations that may be produced during generation of product 88 in method 50 of FIG. 1. The configurations are based on a model of how an exemplary vertebrate telomerase 84 may function and be structured. The vertebrate telomerase adds a hexamer repeat to a telomere in vivo or to a short substrate in vitro. The model is intended to facilitate understanding how product 88 may be generated from a telomerase substrate 86. However, the assays of the present disclosure should to be limited to any theory of structure and operation for telomerase 84.

An active copy of telomerase 84 may include a protein component 162 and an RNA component 164 associated with one another. Protein component 162 may be described as a reverse transcriptase. RNA component 164, which is shown in FIG. 5 in fragmentary form, may serve as an RNA template for the reverse transcriptase activity of the protein component. The RNA template may determine addition of a telomere repeat to the end of substrate 86 (and/or a telomerase-extended intermediate therefrom). The RNA component also may provide a binding site for the 3′-end region of a molecule of substrate 86 (or an extended product molecule).

The top portion of FIG. 5 shows a reaction mixture 80 containing a copy of telomerase 84 and a copy of substrate 86. The telomerase and substrate are not yet associated with each other.

Substrate 86 then may associate with telomerase 84, at least in part via base pairs formed between the 3′-end region of substrate 86 and a sequence region of RNA component 164. For example, here, the last three nucleotides (5′-GTT-3′) at the 3′-end of substrate 86 form base pairs with a trio of nucleotides (3′-CAA-S′) of RNA component 164.

Telomerase 84 then uses its reverse transcriptase activity to extend substrate 86, which generates intermediate product 166 containing the substrate sequence (in upper case) and an appended sequence 168 (in lower case; 5′-agggttag-3′) joined to the substrate at its 3′ end. The appended sequence is templated by RNA component 164. No “C” nucleotides are templated by the RNA component. Accordingly, dCTP can be omitted from the reaction mixture, to reduce undesired side reactions.

Intermediate product 166 then may “slide” along RNA component 164 to shift the base-paired position of the intermediate product. More particularly, the last five nucleotides (5′-gttag-3′) at the 3′-end of intermediate product 166 may form base-pairs with five nucleotides (3′-CAAUC-5′) of the RNA component.

Protein component 162 of telomerase 84 then may extend intermediate product 166 to add six nucleotides (5′-ggttag-3′) templated by RNA component 164. The process of shifting position followed by extension may be repeated any suitable number of times, to add a tandem array of a basic hexamer repeat (5′-ggttag-3′) to generate final product 88. The length of the final product generally varies among the partitions.

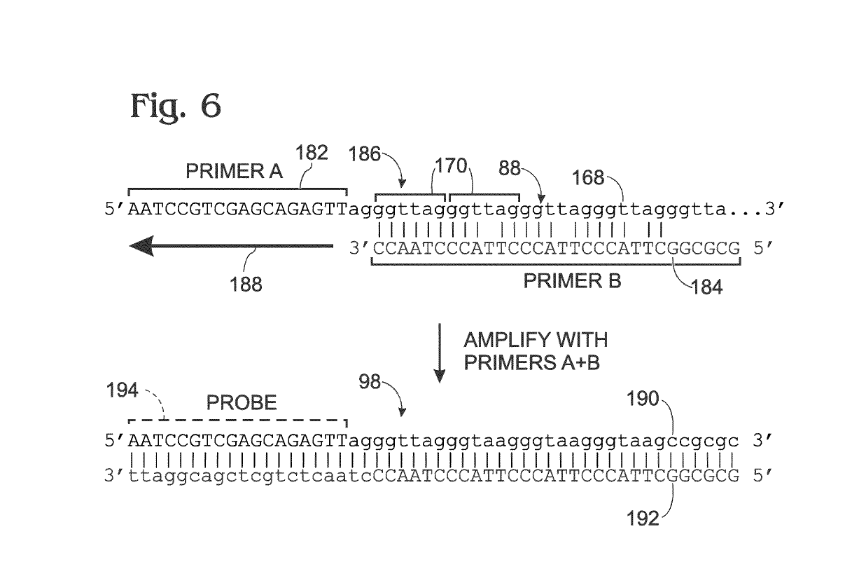

FIG. 6 shows an amplification scheme for amplifying product 88 of FIG. 5 with a pair of primers 182, 184. Primer 182 (primer A) may have a sequence that corresponds substantially to substrate 86 (see FIG. 5). For example, primer 182 may be identical to substrate 86, or may, for example, have at least six or eight consecutive nucleotides in common with the substrate. Primer 184 (primer B) may be configured to bind to a plurality of repeats 170 added by telomerase (see FIG. 5). For example, here, primer 184 binds to about four repeats 170 with three mismatches. Since product 88 has a highly repetitive structure, primer 184 has a plurality of offset options for binding extendably to the product. One potential annealed configuration 186 is shown in FIG. 6. In other embodiments, the 3′-end of primer 184 may base-pair with the integral substrate sequence of the product, such that primer 184 only can be extended at one binding position along product 88. In any event, polymerase extends primer 184, indicated by an arrow at 188, to generate a flush end of the duplex.

Amplification primers 182, 184 then may be utilized to form amplicon 98. The amplicon may include an upper strand 190 that contains the sequence for primer 182 and also contains a sequence region complementary to the full sequence of primer 184. The amplicon also may include a lower strand 192 that contains the sequence for primer 184 and a sequence region complementary to the full sequence of primer 182. The amplicon may have a fixed length or a variable length in any given partition, depending, for example, on the conditions used for amplification, the number of product molecules present initially in the partition before amplification, and the like.

Amplification may be detected via a reporter present during amplification. For example, the reporter may be a probe 194 having nucleotide sequence identity or sequence complementarity to at least a region of one or both primers. The probe may include an oligonucleotide having at least six or at least eight consecutive nucleotides in common with (or complementary to) at least one of the primers and/or in common with (or complementary to) the substrate. Alternatively, the reporter may be a generic reporter for nucleic acid (such as double-stranded nucleic acid) and may bind DNA fairly non-specifically.

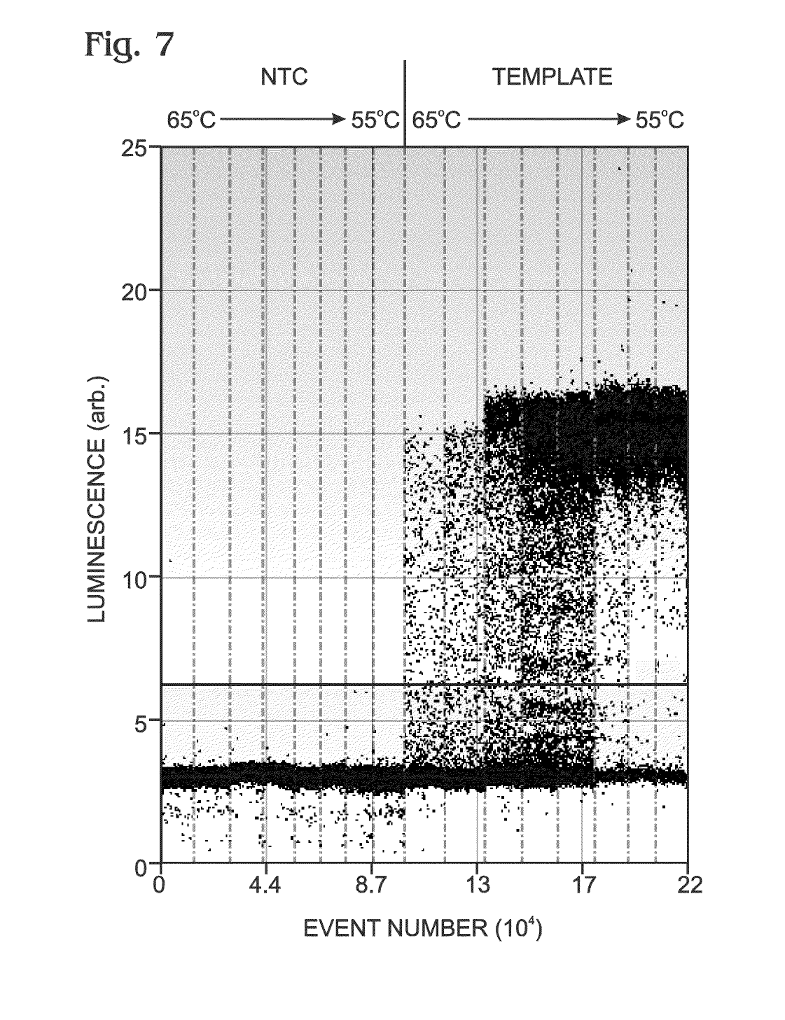

FIG. 7 shows a plot of exemplary photoluminescence data (fluorescence amplitude) collected from droplets after PCR amplification in the droplets from a template (or a no-template control (NTC)) with the primers of FIG. 6 and various annealing temperatures. The template was synthesized chemically, is structured like an extended substrate, and has the sequence 5′-AATCC GTCGA GCAGA GTTAG TTAGG GTTAG GGTTA GGGTT AGGGT TAG-3′. A reaction mixture was formed containing 200 nM of each primer, template at about 3000 copies per μL, dNTPs, Taq DNA Polymerase, and buffer. The reaction mixture was partitioned to form droplets, with the template present at limiting dilution. Droplets were thermally cycled at the indicated annealing temperatures to promote amplification, and fluorescence was measured from individual droplets. The graph of FIG. 7 plots luminescence amplitude, namely, fluorescence intensity in arbitrary units, with respect to time (event number).

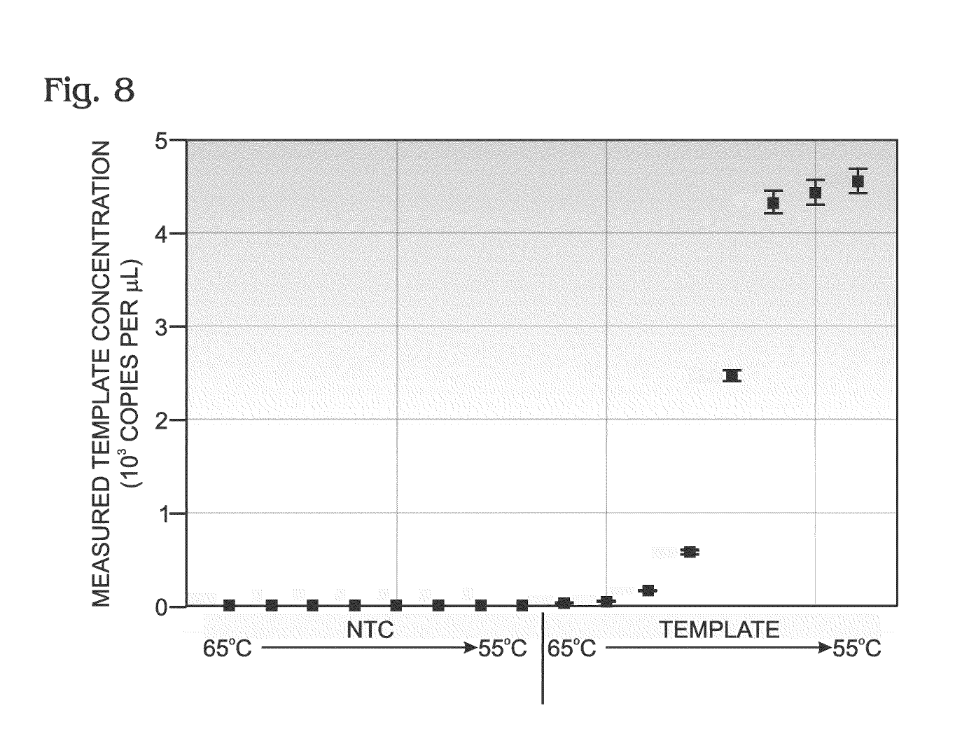

FIG. 8 shows template concentrations calculated from the data of FIG. 7. The template concentration calculated generally plateaus at an annealing temperature of about 57° C. or lower.

### Example 2

### Selected Embodiments

This example describes selected embodiments of an exemplary digital assay system for telomerase, presented as a series of numbered paragraphs.

1. A method of assaying telomerase, the method comprising: (A) creating contact between a sample and a telomerase substrate; (B) generating a product by extending the substrate with telomerase provided by the sample; (C) forming partitions; (D) amplifying an amplicon in the partitions, the amplicon representing the product; (E) collecting data for amplification of the amplicon; and (F) determining a level of the product based on the data.

2. The method of paragraph 1, wherein the level is a concentration of the product in the partitions.

3. The method of paragraph 1, further comprising a step of determining a level and/or activity of telomerase based on the level of the product.

4. The method of paragraph 3, wherein the level and/or activity of telomerase is a level and/or activity in the sample.

5. A method of assaying telomerase, the method comprising: (A) creating contact between a sample and a telomerase substrate; (B) generating a product by extending the substrate with telomerase provided by the sample; (C) forming partitions; (D) amplifying an amplicon in the partitions, the amplicon representing the product; (E) collecting data for amplification of the amplicon; and (F) determining a concentration and/or activity level of telomerase based on the data.

6. The method of paragraph 5, wherein the step of forming partitions is performed after the step of generating a product.

7. The method of paragraph 5, wherein the step of forming partitions is performed before a majority of the product is generated.

8. The method of paragraph 5, wherein the step of generating a product is performed in a reaction mixture, further comprising a step of heating the reaction mixture to inactivate telomerase after the product is formed and before the step of forming partitions.

9. The method of paragraph 5, wherein the step of generating a product is performed in the absence of dCTP.

10. A method of assaying telomerase, the method comprising: (A) forming a reaction mixture containing a sample and a telomerase substrate; (B) generating a product in the reaction mixture by extending the substrate with telomerase provided by the sample; (C) forming partitions containing the product at limiting dilution; (D) amplifying an amplicon in the partitions, the amplicon representing the product; (E) collecting data for amplification of the amplicon; and (F) determining a level of telomerase in the sample based on the data.

11. The method of paragraph 10, wherein the sample is obtained from a source having telomeres structured as a basic repeat, and wherein the substrate includes a primer having at least part of the basic repeat at a 3′-end of the primer.

12. The method of paragraph 10, wherein the step of amplifying is performed with a primer having the same sequence as the substrate.

13. The method of paragraph 10, wherein the step of amplifying is performed with a primer configured to prime substantially better on the product than the substrate.

14. The method of paragraph 10, wherein the partitions include a reporter comprising a luminophore-labeled oligonucleotide.

15. The method of paragraph 14, wherein the oligonucleotide has substantial sequence identity with the substrate.

16. The method of paragraph 15, wherein the oligonucleotide has a region of at least eight contiguous nucleotides that is identical to a region of the substrate.

17. The method of paragraph 14, wherein the oligonucleotide has complementarity to the substrate.

18. The method of paragraph 14, wherein oligonucleotide is capable of forming at least six contiguous base-pairs with the substrate.

19. The method of paragraph 14, wherein the oligonucleotide is labeled with an energy transfer pair including a luminescence donor and a luminescence acceptor.

20. The method of paragraph 19, wherein the luminescence acceptor is a quencher.

21. The method of paragraph 10, wherein the sample is a cell lysate.

22. The method of paragraph 10, wherein the reaction mixture is combined with reagents for the step of amplifying before the partitions are formed.

23. The method of paragraph 10, wherein the step of collecting data includes a step of detecting photoluminescence from the partitions.

24. The method of paragraph 10, wherein the reporter includes a dye that binds DNA at least generally non-specifically.

25. The method of paragraph 24, wherein the dye is an intercalating dye.

26. The method of paragraph 10, wherein the step of generating a product is performed with the reaction mixture maintained at a temperature of less than about 40° C.

27. The method of paragraph 26, wherein the step of generating a product is performed with the reaction mixture maintained at a constant temperature.

28. The method of paragraph 10, wherein the level is a level of telomerase activity exhibited by telomerase in the sample.

29. The method of paragraph 10, wherein the partitions are droplets.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure. Further, ordinal indicators, such as first, second, or third, for identified elements are used to distinguish between the elements, and do not indicate a particular position or order of such elements, unless otherwise specifically stated.

We claim:

1. A method of assaying telomerase, the method comprising:
generating a product by extending a substrate with telomerase;
forming partitions containing at least a portion of the telomerase;
performing an amplification reaction in partitions with the product as a template;
collecting data for the amplification reaction from a plurality of the partitions; and
determining a level of the product based on the data.

2. The method of claim 1, wherein the product and/or telomerase is present at limiting dilution in the partitions before the amplification reaction is started.

3. The method of claim 1, further comprising a step of determining a presence or activity of telomerase based on the level of the product.

4. The method of claim 3, wherein the step of determining a presence or activity of telomerase includes a step of determining a concentration of telomerase.

5. The method of claim 3, wherein the step of determining a presence or activity of telomerase is based on an assumption that each active copy of telomerase generates only one molecule of the product.

6. The method of claim 1, wherein the step of generating a product is performed at least in part before the step of forming partitions.

7. The method of claim 1, further comprising a step of heating at least a portion of the telomerase after the product is generated to inactivate the at least a portion of the telomerase before the step of forming partitions.

8. The method of claim 1, wherein the step of generating a product is performed without dCTP.

9. A method of assaying telomerase, the method comprising:
generating a product by extending a substrate with telomerase;
forming partitions containing the product at limiting dilution;
performing an amplification reaction in partitions with the product as a template;
collecting data for the amplification reaction from individual partitions; and
determining a presence or activity of telomerase based on the data.

10. The method of claim 9, wherein the step of determining includes a step of determining a concentration of telomerase.

11. The method of claim 9, wherein the step of forming partitions includes a step of forming droplets.

12. The method of claim 9, wherein the substrate is an oligonucleotide.

13. The method of claim 9, wherein telomerase for the step of generating a product is provided by a cell lysate.

14. The method of claim 9, wherein telomerase for the step of generating a product is provided by a sample obtained from a source having telomeres structured as a basic repeat, and wherein the substrate includes a primer having at least two nucleotides of the basic repeat at a 3′-end of the primer.

15. The method of claim 9, wherein the amplification reaction is performed with a primer having a sequence of at least six consecutive nucleotides in common with substrate.

16. The method of claim 9, wherein the partitions include a photoluminescent reporter comprising an oligonucleotide.

17. The method of claim 9, wherein the partitions include a generic reporter for nucleic acid.

18. The method of claim 17, wherein the generic reporter includes an intercalating dye.

19. The method of claim 9, wherein the step of determining a presence or activity of telomerase includes a step of counting partitions deemed to be positive for the amplification reaction and/or a step of counting partitions deemed to be negative for the amplification reaction.

20. The method of claim 9, wherein the step of generating a product is performed in a reaction mixture, further comprising a step of combining the reaction mixture with one or more reagents for the step of performing an amplification reaction, after the step of generating a product and before the partitions are formed.
